(19) United States
(12) Patent Application Publication
(10) Pub. No.: US 2022/0096089 A1
(43) Pub. Date: Mar. 31, 2022

(54) CARTRIDGE, AND TREATMENT DEVICE FOR ENDOSCOPE COMPRISING SAME

(71) Applicant: TAEWOONG MEDICAL CO., LTD., Gyeonggi-do (KR)

(72) Inventors: Kyong Min SHIN, Gyeonggi-do (KR); Byung Chol KIM, Seoul (KR)

(73) Assignee: TAEWOONG MEDICAL CO., LTD., Gyeonggi-do (KR)

(21) Appl. No.: 17/427,067

(22) PCT Filed: Jan. 31, 2020
(86) PCT No.: PCT/KR2020/001454

§ 371 Date: Jul. 29, 2021

(30) Foreign Application Priority Data
Feb. 1, 2019 (KR) 10-2019-0013268
Jul. 5, 2019 (KR) 10-2019-0081388

(51) Int. Cl. A61B 17/122; A61B 17/00

(52) U.S. Cl. CPC A61B 17/122; A61B 2017/00296; A61B 17/00234

(57) ABSTRACT

Provided are a cartridge, and a treatment device for an endoscope comprising same. The present invention includes a straight guide path in which a clip connector and a moving tube separation bar are arranged in a straight line, and arranges a front moving tube, which is inserted in and arranged on the tip of a clip facing the moving tube separation bar and temporarily restrains a pair of forceps in a retracted state, and arranges a rear moving tube, which is assembled on the outer surface of the clip connector, so as to be slidably movable along the straight guide path toward the moving tube separation bar, wherein the clip is restrained in a retracted state inside an insertion tube entering internally along the straight guide path.

### TECHNICAL FIELD

The present invention relates to a cartridge which accommodates a clip that is temporarily restricted while being retracted and a clip connection body and an endoscopic treatment device used for endoscopic treatment.

### BACKGROUND ART

Recently, a therapeutic endoscopy field using an endoscope has been accepted as a significant field in a digestive system medical treatment area. A medical endoscope using an electron microscope, that is, endoscopic treatment, has been developed from the appearance of a micro solid state image sensing device to miniaturization of an electronic device, development of imaging technology, a variety of diagnoses using a working channel, development of therapy, and maneuvering skill of a doctor in charge.

For example, although cases of stomach cancer or colorectal cancer depended on surgery in the past, recently with the appearance of an endoscope, diagnosis and therapy are clinically performed at the same time by performing endoscopic submucosal dissection (ESD) through a treatment device inserted into a working channel provided in an endoscope so that an endoscopic cure rate of early gastic cancer, early colorectal cancer, or the like has increased.

There are a variety of types of treatment tools used for a treatment device inserted through a working channel of an endoscope when ESD or endoscopic mucosal resection (EMR) is performed.

That is, a treatment device is used while entering a body through a tube connected to a treatment body included in the treatment device and inserted into a working channel provided in an endoscope. Generally, a variety of types of treatment devices are used such as a knife generally used for mucosectomy of tissue, a coagulator used to stop bleeding during a mucosectomy and the like, a needle for injecting medication necessary for injecting a saline solution or the like for swelling tissue to provide ease in surgical removal, and injection of a washer fluid for ease in securing a field of vision against bleeding or the like.

Patent Document 1: KR10-1793631 B1

Patent Document 1 discloses an endoscopic treatment device in which a separation bar including a fixed body connected to the other end of the separation bar having a front end extending toward a first accommodation portion and fixed in position to a third accommodation portion is included so that an external moving tube coming into contact with an insertion tube and forcibly moved passes through a stopper and is located in a clip so as to convert the clip in the first accommodation portion into a retracted state, to elastically link and connect a linker to a second connector of a clip connection body while a front end of the clip converted into the retracted state in the first accommodation portion comes into contact with an end of the separation bar, and to move and separate the external moving tube located in the clip to the separation bar.

Also, in Patent Document 1, the clip disposed and standing by in a spreading state in the first accommodation portion having a diamond shape frequently causes a failure in use in which serration portions of the front end of the clip that join when being retracted do not accurately come into contact with a center of the end of the separation bar.

That is, a product defect is caused in which, as the serration portions that are ends of the clip converted from the spreading state into the retracted state do not come into accurate contact with the separation bar and stop but deviate or slide outward, and thus a user cannot perform an operation of linking and connecting the liner to the clip connection body while the clip is stopped such as to degrade a reliability of product.

Also, a second contact guide surface of the first accommodation portion in which the clip is disposed is formed as a tilted surface having a path gradually narrowing toward the third accommodation portion in which the separation bar is disposed so that the clip moved toward the separation bar while coming into contact with the second contact guide surface is converted into being retracted.

Here, since a planar end of the separation bar is spaced at a certain interval apart from a terminal end of the second contact guide surface and an additional guide structure guiding to the front end of the separation portion is not present, the serration portions that are the front end of the clip converted into the retracted state while moving in contact with the second contact guide surface may not come into accurate contact with a center of the planar end of the separation bar and may deviate or slide outward.

Particularly, the serration portions being bent at an obtuse angle that are front end of the clip act as a major cause increasing frequency of a failure in which the front end of the clip having a pointed shape does not come into contact with the center of the planar end of the separation bar and stop but deviates or slides.

Also, since it is necessary to secure a movement section for converting the clip in an initial state of spreading into the retracted state in the cartridge, there is a limitation in reducing a length and a volume of the cartridge and a manufacturing cost of a product mold is increased to precisely form a diamond-shaped contact guide section.

### DISCLOSURE

### Technical Problem

The present invention is directed to providing a cartridge configured to accurately guide a front end of a clip waiting to be retracted to a set stop position and to perform an operation of linking and connecting a linker to a clip connection body without a failure when the clip is stopped and an endoscopic treatment device including the cartridge.

The technical aspects achievable by the present invention are not limited to the above-stated aspects and other unstated aspects may be clearly understood by one of ordinary skill in the art from the following disclosure.

### Technical Solution

One aspect of the present invention provides a cartridge including a clip including a pair of tong pieces, a clip connection body having one end held by and connected to the clip, and a moving tube separation bar facing the clip. The cartridge includes a linear guide path on which the clip connection body and the moving tube separation bar are linearly aligned and arranged. Here, a front moving tube inserted and disposed in a front end of the clip facing the moving tube separation bar and configured to temporarily restrict the pair of tong pieces in a retracted state and a rear moving tube assembled with an outer surface of the clip connection body are arranged to be slidable and movable along the linear guide path toward the moving tube separation bar. Also, the clip is restricted again in the retracted state in the insertion tube entering along the linear guide path.

The front moving tube may be disposed on the linear guide path while a front end thereof is partially inserted into an end of the moving tube separation bar or may be disposed on the linear guide path while being spaced at a certain interval apart from the end of the moving tube separation bar.

The moving tube separation bar may include a notch portion recessively formed in an end corresponding to the front end of the clip retracted by the front moving tube.

The moving tube separation bar may include a separation sensing portion configured to generate a frictional force while outer surfaces and inner surfaces of the front moving tube and the rear moving tube passing therethrough, and a column portion in which the front and rear moving tubes passing through the separation sensing portion are disposed.

Another aspect of the present invention provides an endoscopic treatment device including an insertion tube including a wire including a linker on a front end, a clip including a pair of tong pieces having front ends bent to face each other and intersecting with each other, a clip operating portion including a clip connection body having one end held by and connected to the clip and a stopper with which the clip connection body is assembled to be reciprocable therein, and a cartridge including a linear guide path, inside a body thereof, on which the clip connection body and a moving tube separation bar are linearly aligned and arranged. Here, the endoscopic treatment device includes a front moving tube inserted and disposed in a front end of the clip facing the moving tube separation bar and configured to temporarily restrict the pair of tong pieces in a retracted state and a rear moving tube assembled with an outer surface of the clip connection body. The front moving tube and the rear moving tube are allowed to slide and move by an external force of the insertion tube entering the cartridge along the linear guide path so as to be separately collected by the moving tube separation bar. Also, the linker and the clip connection body are link-connected to each other while the front end of the clip restricted again in the retracted state in the insertion tube comes into contact with an end of the moving tube separation bar.

The linker may include a first linker and a second linker adjacent thereto, and the clip connection body may include a first connector held by and connected to the clip and a second connector link-connected to the linker.

The second connector may include a pair of elastic holding pieces divided by a slit line at an end corresponding to the first linker and a hollow portion in which the first and second linkers entering to elastically spread the pair of elastic holding pieces are inserted and disposed. Also, the first linker may be held by and connected to an internal step protruding from an inner surface of the hollow portion and the second linker may be held by and connected to a protruding portion protruding from inner ends of the pair of elastic holding pieces.

The second linker may include a concave groove forming the protruding portion and a tilted contact boundary surface in a rear surface corresponding to the protruding portion.

Still another aspect of the present invention provides an endoscopic treatment device including an insertion tube including a wire including a linker on a front end, a clip including a pair of tong pieces having front ends bent to face each other and intersecting with each other, a clip operating portion equipped with a clip connection body including a connection bar configured to connect a first connector having one end held by and connected to the clip to a second connector having the other end link-connected to the linker so as to spread or retract the clip, and a cartridge including a linear guide path, inside a body thereof, on which the clip connection body and a moving tube separation bar are linearly aligned and arranged. Here, the second connector includes a pair of elastic holding pieces divided by a slit line at an end corresponding to the linker and a hollow portion in which the linker entering to elastically spread the pair of elastic holding pieces is inserted and disposed. Also, the linker is held by and connected to an internal step protruding from an inner surface of the hollow portion or is held by and connected to a protruding portion protruding from inner ends of the pair of elastic holding pieces.

The linker may include a first linker and a second linker adjacent thereto.

The second connector may include a pair of elastic holding pieces divided by a slit line at an end corresponding to the linker and a hollow portion in which the linker entering to elastically spread the pair of elastic holding pieces is inserted and disposed. Also, the first linker may be held by and connected to an internal step protruding from an inner surface of the hollow portion or the second linker may be held by and connected to a protruding portion protruding from inner ends of the pair of elastic holding pieces.

The linker may include a concave groove forming the protruding portion and a tilted contact boundary surface in a rear surface corresponding to the protruding portion.

The linker may enter, to a certain length, inward from an open inlet end of the insertion tube and may stand by therein.

The endoscopic treatment device may include a first incised portion incised and formed to externally expose the linker to one side in an outer surface of one side of the second connector and a second incised portion incised and formed to extend from the slit line and give an elastic force to the pair of elastic holding pieces in an outer surface of the other side of the second connector.

Yet another aspect of the present invention provides an endoscopic treatment device including an insertion tube including a wire including a linker on a front end, a clip including a pair of tong pieces having front ends bent to face each other and intersecting with each other, a clip operating portion equipped with a clip connection body including a connection bar configured to connect a first connector having one end held by and connected to the clip to a second connector having the other end link-connected to the linker so as to spread or retract the clip, and a cartridge including a linear guide path, inside a body thereof, on which the clip connection body and a moving tube separation bar are linearly aligned and arranged. The endoscopic treatment device includes a front moving tube inserted and disposed in a front end of the clip facing the moving tube separation bar and configured to temporarily restrict the pair of tong pieces in a retracted state and a rear moving tube assembled with an outer surface of the clip connection body. Here, the second connector includes a pair of elastic holding pieces divided by a slit line on an end corresponding to the linker and a hollow portion in which the linker entering to elastically spread the pair of elastic holding pieces is inserted and disposed The front moving tube and the rear moving tube are allowed to slide and move by an external force of the insertion tube entering the cartridge along the linear guide path so as to be separately collected by the moving tube separation bar. Also, while the front end of the clip restricted again in a retracted state in the insertion tube comes into contact with an end of the moving tube separation bar, the linker is held by and connected to an internal step protruding from an inner surface of the hollow portion or is held by and connected to a protruding portion protruding from inner ends of the pair of elastic holding pieces so as to be link-connected to the clip connection body.

### Advantageous Effects

According to exemplary embodiments of the present invention, the following effects are present.

Since a clip temporarily restricted in a retracted state by a front moving tube and a clip operating portion assembled with a rear moving tube are linearly aligned and arranged on a linear guide path of a cartridge so that the front moving tube and the rear moving tube are forcibly moved to a moving tube separation bar and separated by an external force of an insertion tube entering along the linear guide path and a front end of the clip restricted again in the retracted state in the insertion tube is accurately stopped at an end of the moving tube separation bar, an operation of linking and connecting a linker to a clip connection body may be accurately and safely performed in the insertion tube entering the cartridge so as to increase a reliability of product and increase satisfaction of a user.

Since whether the front and rear moving tubes disposed on a column portion of the moving tube separation bar via the clip are removed may be simply and accurately seen without additional components by including a separation sensing portion in the moving tube separation bar, where the front moving tube and the rear moving tube pass through and come into contact with the separation sensing portion, the following operations of linking and connecting the clip connection body to the linker in the cartridge and separating and withdrawing the insertion tube from the cartridge may be continuously performed and convenience in use may be increased.

Since the linker including first and second linkers is inserted and disposed in a hollow portion formed in the clip connection body so that the linker is held by and connected to an internal step formed in the hollow portion while being held by and connected to a protruding portion formed on the elastic holding piece so as to increase a link-connection force between the clip connection body and the linker, product reliability may be increased by basically preventing a defect in which the linker is separated from the clip connection body when the clip is spread and retracted by forward and backward movement of a wire.

### DESCRIPTION OF DRAWINGS

FIG. 1 is an external perspective view of an endoscopic treatment device according to an embodiment of the present invention.

FIG. 2 is a perspective view illustrating a state in which a clip, a clip operating portion, and a moving tube separation bar are aligned and arranged on a cartridge of the endoscopic treatment device according to the embodiment of the present invention.

FIG. 3 is an exploded perspective view of the endoscopic treatment device according to the embodiment of the present invention.

FIGS. 4A and 4B are assembling configuration diagrams illustrating the clip, the clip operating portion, and a linker of the endoscopic treatment device according to the embodiment of the present invention.

FIG. 5 is a detailed view illustrating a coupled state of the clip, a clip connection body, and the linker of the endoscopic treatment device according to the embodiment of the present invention.

FIGS. 6A and 6B are cross-sectional perspective views illustrating a connected and coupled state of the clip connection body and the linker of the endoscopic treatment device according to the embodiment of the present invention.

FIGS. 7A, 7B, and 7C are configuration diagrams illustrating an implemented shape of the moving tube separation bar applied to the endoscopic treatment device according to the embodiment of the present invention.

FIGS. 8A, 8B, 8C, 8D, 8E, and 8F illustrate a process of using the endoscopic treatment device according to the embodiment of the present invention.

### MODES OF THE INVENTION

An exemplary embodiment of the present invention which may be easily implemented by one of ordinary skill in the art will be described in detail with reference to the attached drawings. However, in a detailed description of a structural principle with respect to the exemplary embodiment of the present invention, a detailed description of well-known functions or components of the related art will be omitted when it is deemed to unnecessarily obscure the gist of the present invention.

Also, throughout the drawings, parts configured to have similar functions and operations will be referred to as the same reference numerals.

In addition, throughout the specification, when a part is stated as being “connected” to another part, the part may not only be “directly connected” but may also “indirectly connected” to the other part with another device therebetween. Also, “comprising” another component means, unless particularly disclosed otherwise, further comprising another component instead of excluding the other component.

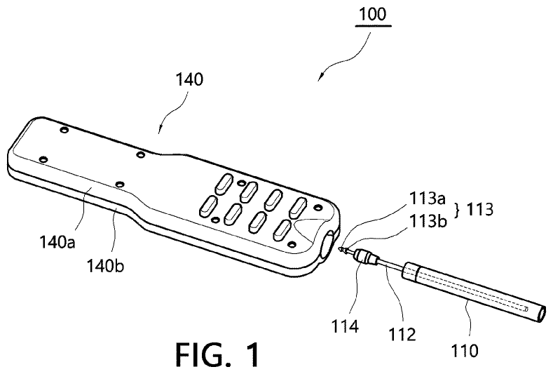

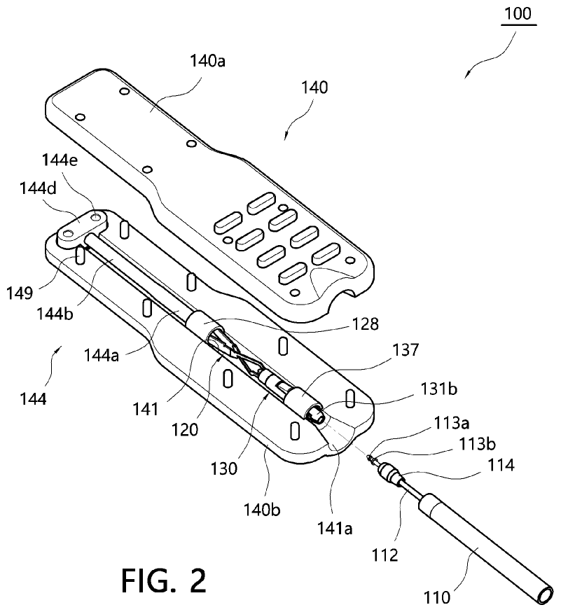

An endoscopic treatment device 100 according to an exemplary embodiment of the present invention includes, as shown in FIGS. 1, 2, and 3, an insertion tube 110 including a linker 113, a clip 120 including a pair of tong pieces 121a and 121b, a clip operating portion 130 including a clip connection body 131 connected to the clip 120, and a cartridge 140 which accommodates the above components therein.

As shown in FIGS. 1, 2, and 3, the insertion tube 110 includes a hollow tube having a certain length and a wire 112 having a certain length which is a manipulating wire inserted and disposed to reciprocate back and forth through an inner hollow of the hollow tube in a longitudinal direction due to an external force so as to be inserted into a channel of an endoscope in surgery.

The insertion tube 110 may be formed of a flexible material to be naturally curved along a curved portion formed in the endoscope.

The linker 113 elastically link-connected to a rear end of the clip connection body 131 having a front end connected to the clip 120 is formed on a front end of the wire 112.

The linker 113 includes a first linker 113a having an approximate conic cross-sectional shape corresponding to the clip connection body 131 and includes a second linker 113b having a conic cross-sectional shape approximately equal to the first linker 113a and provided to be adjacent to the first linker 113a.

Here, although the first linker 113a is shown and described as having a conic cross-sectional shape having an outer diameter smaller than an outer diameter of the second linker 113b, the present invention is not limited thereto and the first linker 113a and the second linker 113b may have conic cross-sectional shapes having the same outer diameter.

Behind the second linker 113b, an insertion body 114 formed to have an approximate cylindrical shape, be disposed inside the insertion tube 110, and be inserted into the cartridge with the linker 113 is provided.

The first linker 113a, the second linker 113b, and the insertion body 114 are formed as an integral structure and integrally provided on the front end of the wire 112.

Here, the insertion tube 110 may be provided to be sufficiently longer than a length capable of being inserted into and passing through a treatment device insertion through channel of the endoscope, and the insertion body 114 may be formed to have a tilted surface with an outer diameter being decreased on a part of a front end to easily enter the cartridge.

At least one grip member (not shown) having a front end integrally connected to the insertion body and disposed on an outer surface of the wire may be provided on a rear end of the insertion body 114 to allow a user to easily grip the wire and to prevent slipping while the user handles the wire.

The grip member may be formed of a coil-shaped spring member having an outer diameter relatively smaller than an inner diameter of the insertion tube 110 and may be welded to the insertion body 114.

A rear end of the wire 112 having the front end on which the linker 113 is provided is connected to a manipulating handle (not shown) to manipulate, back and forth, the wire 112 disposed inside the insertion tube.

Meanwhile, although the linker 113 is shown and described as being formed of a pair of members including the first linker 113a in front thereof and the second linker 113b in the rear, the present invention is not limited thereto and the linker may be formed as a single member including only the second linker 113b in the rear and link-connected to the clip connection body 131.

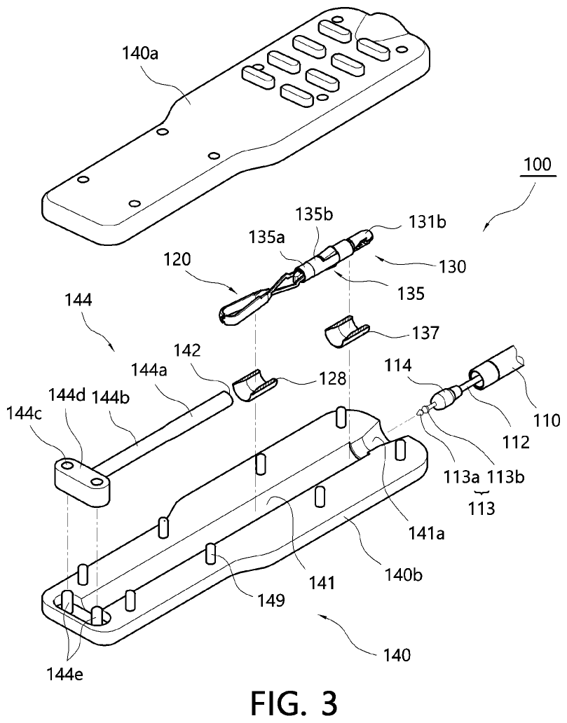

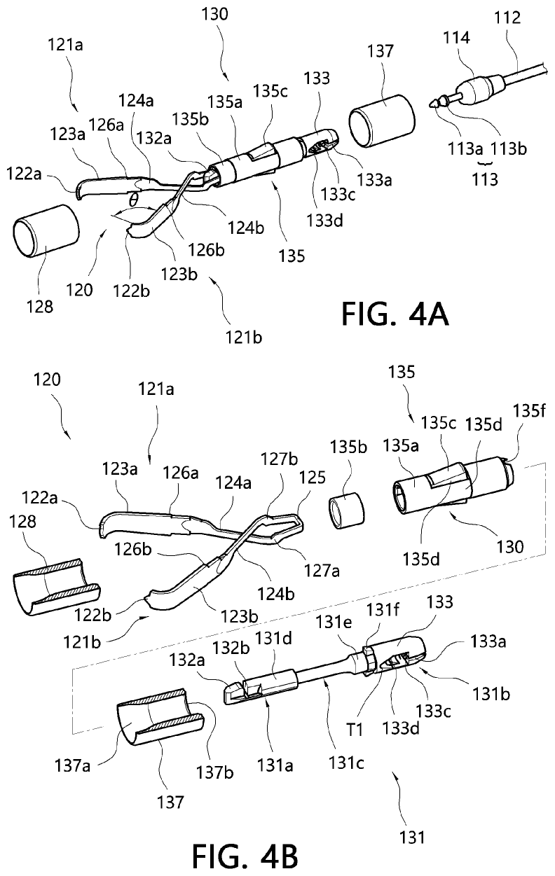

As shown in FIGS. 3, 4A, and 4B, the clip 120 is bent so as to allow front ends thereof to face each other, includes the pair of tong pieces 121a and 121b intersecting with each other in the middle of length to be held by and connected to the clip connection body 131 of the clip operating portion 130, and is accommodated with the clip operation portion 130 in a linear guide path 141 of the cartridge 140 while being retracted.

In the clip 120, the linker 113 and the clip connection body 131 are link-connected to each other in the insertion tube 110 entering along the linear guide path 141 of the cartridge 140 and then the clip 120 and the clip operating portion 130 are separated and withdrawn together from the inside of the cartridge 140 to the outside with the insertion tube.

The pair of tong pieces 121a and 121b include tong plates 123a and 123b having serration portions 122a and 122b on one ends bent to face each other, a pair of intersecting plates 124a and 124b extending, by a certain length, from other ends of the tong plates 123a and 123b and intersecting with each other, and a connection plate 125 integrally connecting one ends of the intersecting plates 124a and 124b intersecting with each other.

The serration portions 122a and 122b are provided on ends of the pair of tong plates 123a and 123b to face each other to be engaged with each other when being retracted according to proximity between the pair of tong pieces 121a and 121b and to generate a grip force for removing a part of human anatomy in surgery using the endoscope.

The tong plates 123a and 123b may be formed to have an approximate arc-shaped cross-sectional shape to prevent an inner surface of the channel from being damaged by frictional resistance when entering the channel of the endoscope having an approximate circular hole cross-sectional shape.

The intersecting plates 124a and 124b are formed of plate members having a width that is relatively smaller than a plate width of the tong plates 123a and 123b and intersect with each other, and the connection plate 125 may be formed of a plate member having an approximate staple-shaped cross-sectional shape bent from ends of the intersecting plates 124a and 124b and connecting the intersecting plates.

Here, widths of the intersecting plates 124a and 124b may be provided as approximately half the tong plates 123a and 123b to allow the pair of tong pieces 121a and 121b to spread and be retracted more elastically.

Since first holding steps 126a and 126b with which a front end of a stopper 135 of the clip operating portion 130 comes into contact are formed on a boundary area between the pair of tong pieces 123a and 123b and the intersecting plates 124a and 124b to protrude outward, a retracted state of the pair of tong plates 123a and 123b may be maintained when the first holding steps 126a and 126b come into contact with a second stopper tube body 135b, that is, the front end of the stopper 135 that is moved to the pair of tong plates 123a and 123b.

Since second holding steps 127a and 127b with and by which the stopper 135 is interfered and held are formed to protrude outward in a boundary area between the connection plate 125 and the intersecting plates 124a and 124b, a maximal spreading state of the tong plates 123a and 123b may be maintained when the second holding steps 127a and 127b come into contact with the second stopper tube body 135b that is the front end of the stopper 135 pushed and moved to the tong plates 123a and 123b.

Here, since the serration portions 122a and 122b are formed to be bent at a certain angle to face front ends of the pair of tong plates 123a and 123b, a bending angle θ formed between the tong plate and the serration portion may be about 90° or may be an obtuse angle greater than 90°.

The front ends of the pair of serration portions 122a and 122b which come into contact with each other when the pair of tong pieces 121a and 121b are retracted may be shape-matched in a panel shape or shape-matched in a pointed protruding shape according to a size of the bending angle θ so as to generate a clamping force for removing a part of human anatomy in surgery.

The clip 120 includes at least one front moving tube 128 inserted into and disposed in the front ends of the pair of tong pieces 121a and 121b retracted to have an elastic restoration force to spread outward to temporarily restrict the pair of tong pieces in the retracted state.

The front moving tube 128 is formed of a hollow cylindrical tube body configured to temporarily restrict the pair of tong pieces 121a and 121b in the retracted state in the cartridge 140 until the linker 113 and the clip connection body 131 are link-connected to each other.

Here, an inner diameter of the front moving tube 128 restricting the pair of retracted tong pieces 121a and 121b may be a size capable of maintaining a state in which the pair of serration portions 122a and 122b, which are front ends of the pair of retracted tong pieces, come in contact with each other.

The clip 120 that is temporarily restricted in the retracted state by the front moving tube 128 is aligned and disposed in the linear guide path 141 provided linearly inside the cartridge 140 with the clip operating portion 130.

The front moving tube 128 slides and moves along the linear guide path 141 of the cartridge 140 to a moving tube separation bar 144 with a rear moving tube 137 which will be described below and is moved forward to the clip 120 due to an external force of the insertion tube 110 entering the cartridge 140 so as to be completely separated from the clip and to release a temporarily restricting force on the clip.

The clip operating portion 130 includes the clip connection body 131, the stopper 135, and the rear moving tube 137 as shown in FIGS. 3, 4A, 4B, and 5.

The clip operating portion 130 is a structure accommodated in the cartridge 140 like the clip 120, in which the clip connection body 131 and the linker 113 are link-connected to each other inside the insertion tube 110 entering the linear guide path 141 of the cartridge 140 and then are withdrawn with the insertion tube 110 and the clip 120 from the inside of the cartridge 140 to the outside.

The clip operating portion 130 that is withdrawn and separated outward from the cartridge 140 is a structure configured to perform a function of spreading or retracting the clip 120 including the pair of tong pieces 121a and 121b outward or inward in connection with back-and-forth reciprocation of the wire 112 inside the insertion tube.

The clip connection body 131 is a connection member including a first connector 131a where the connection plate 125 that is a rear end of the clip 120 is held and connected, on a front end that is one end, and a second connector 131b where the first and second linkers 113a and 113b included in the linker 113 are elastically inserted to be held and connected, on a rear end that is the other end, so as to connect the clip 120 to the linker 113.

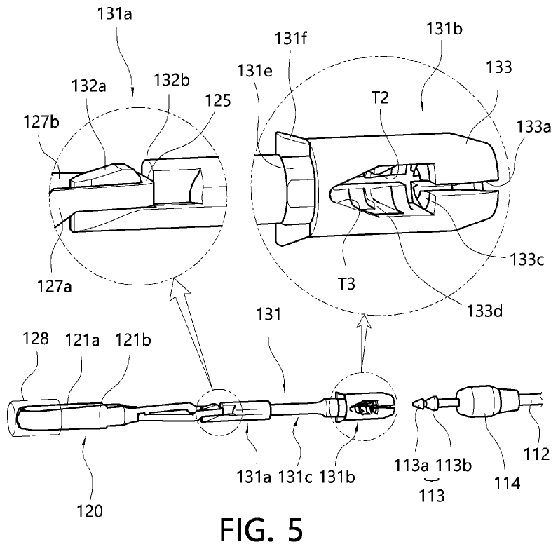

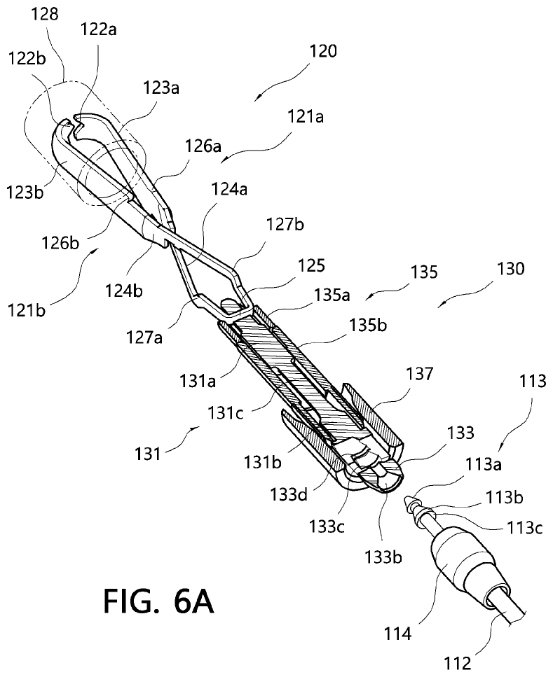

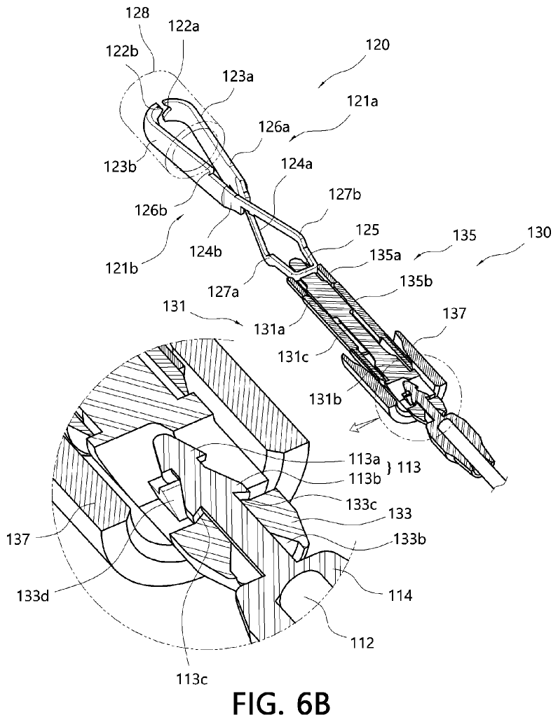

The first connector 131a includes a holding step 132a forming a concave groove 132b to hold and dispose the connection plate 125 provided on rear ends of the tong pieces 121a and 121b as shown in FIGS. 5, 6A, and 6B.

Accordingly, in connection between the clip 120 and the clip connection body 131, when the connection plate 125 of the clip is correspondingly inserted into the concave groove 132b from an open top to a bottom thereof while the first connector 131a that is the front end of the clip connection body 131 inserted through a guide hole of the stopper 135 is exposed outward through the front end of the stopper, the connection plate 125 is held by and disposed in the concave groove 132b. Accordingly, the clip 120 is connected to the holding step 132a of the clip connection body 131, and the clip 120 and the clip connection body 131 are arranged and accommodated in the linear guide path 141 of the cartridge 140 while in this connected state.

Here, in the clip 120 connected to the clip connection body 131, the pair of tong pieces 121a and 121b are restricted in the retracted state by press-fit disposing the front moving tube 128, and the front moving tube 128 restricting the pair of tong pieces 121a and 121b in the retracted state is correspondingly disposed to face a front end of the moving tube separation bar 144.

As shown in FIGS. 5, 6A, and 6B, the second connector 131b includes a pair of elastic holding pieces 133 divided by a slit line 133a that is cut to be elastically link-connected to the linker 113 sequentially including the first and second linkers 113a and 113b in conic cross-sectional shapes.

The second connector 131b includes a hollow portion inside a body, in which the first and second linkers 113a and 113b entering while elastically spreading the pair of elastic holding pieces 133 divided by the slit line 133a are held by and disposed on an end corresponding to the first linker 113a.

Here, a guide groove where the linker 113 including the first and second linkers enters and is guided may be recessively formed in inner surfaces of the elastic holding pieces 133 facing each other, and a tilted guide surface 133b extending outward may be formed on an inlet end of the guide groove to allow the first linker 113a to smoothly perform initial entry.

The first linker 113a inserted and disposed in the hollow portion is primarily held by and connected to an internal step 133d formed to protrude from an inner surface of the hollow portion and the second linker 113b inserted and disposed in the hollow portion with the first linker 113a is secondarily held by and connected to inner ends of the pair of elastic holding pieces 133 so as to link-connect the clip connection body 131 and the linker 113 as one structure.

A protruding portion 133c coming into contact with and being held by and connected to the concave groove 113c recessively formed in a rear surface of the second linker 113b facing the insertion body is formed on inner surfaces of the pair of elastic holding pieces 133 where the second linker 113b is held thereby and connected thereto.

Here, since the protruding portion 133c is formed as an approximately tapered tilted surface to form a tilted contact boundary surface with the concave groove 113c, when the linker 113 is moved rearward by pulling of the wire 112, a contact area between the concave groove of the second linker 113b and the protruding portion 133c of the elastic holding piece 133 increases while an external force is generated in a direction in which the pair of divided elastic holding pieces 133 are retracted inward so that a link-connection state between the linker 113 and the clip connection body 131 may be stably and firmly secured when the wire is manipulated.

A first incised portion T1 incised to externally expose the linker 113 on one side where the first and second linkers are held by and connected to the internal step and protruding portion while being inserted and disposed in the hollow portion may be included on an outer surface of one side of the second connector 131b, and a second incised portion T2 extending from the slit line 133a to provide an elastic force to the pair of elastic holding pieces 133 may be formed in an outer surface of the other side of the second connector 131b to be incised.

Accordingly, when the linker 113 including the first linker 113a and the second linker 113b is inserted into and disposed in the hollow portion formed in the second connector 131b of the clip connection body, the linker 113 and the clip connection body 131 are linked and coupled to each other in the insertion tube entering along the linear guide path 141 of the cartridge 140 through link-connection by holding the first linker 113a and the internal step 133d and link-connection by holding the second linker 113b and the elastic holding piece 133.

The clip 120 and the linker 113 are coupled to each other with the clip connection body 131 as a medium therebetween as one structure in the insertion tube and are reciprocable in the guide hold in the stopper 135 by manipulating the wire provided in the insertion tube to move back and forth.

Also, the clip connection body 131 includes a connection bar 131c having a certain length and configured to integrally connect the first connector 131a that the clip 120 is held by and connected to and the second connector 131b to which the first and second linkers 113a and 113b of the linker are link-connected.

Since the connection bar 131c is provided to have a size that is relatively smaller than an inner diameter of the guide hole passing through the stopper 135 so that frictional resistance caused by contact between an outer surface of the connection bar and an inner surface of the guide hole is not generated, reciprocation of the clip connection body 131 performed in the guide hole of the stopper 135 may be more easily performed.

Also, since an empty space is formed by a gap between the outer surface of the connection bar 131c and the inner surface of the guide hole of the stopper 135, a contractible spare space in which a wing piece 135c protruding from the outer surface of the stopper 135 is retracted inward when the rear moving tube 137 moves forward may be provided and the degree of freedom in design which provides an adequately great blade thickness of the wing piece may be increased to maintain intensity of the wing pieces 135c.

First and second outer diameter portions 131d and 131e having the same outer diameter approximately equal to the inner diameter of the guide hole formed in the stopper 135 to dispose the clip connection body 131 in a center of the guide hole of the stopper 135 to be circumscribed with the inner surface of the guide hole are formed between one end of the connection bar 131c and the first connector 131a and between the other end of the connection bar 131c and the second connector 131b, respectively.

The stopper 135 is a hollow assembled structure including a first stopper tube body 135a in which the first and second outer diameter portions 131d and 131e of the clip connection body 131 are disposed therein and a second stopper tube body 135b disposed in contact with a front end of the first stopper tube body 135a to correspond to a holding connection portion between the clip 120 and the first connector 131a so as to assemble the clip connection body with the guide hole, which is an internal hole, to be reciprocable.

The first stopper tube body 135a includes the pair of wing pieces 135c formed of elastic bodies extending to be tilted so as to spread outward by a certain angle from a pair of incised portions 135d formed in an outer surface in the middle of a length to be incised.

Here, the first stopper tube body 135a forming the pair of wing pieces 135c may include a resin material. The second stopper tube body 135b in which the first connector 131a connected to the clip is disposed may include a resin material or metal material which has durability and strength that are relatively higher than the first stopper tube body 135a including the resin material so as to prevent deformation and abrasion caused by direct contact with the clip when the clip that is externally withdrawn from the cartridge is clamped.

The stopper 135 has been described and shown as independently including the first stopper tube body 135a having a certain length and the second stopper tube body 135b having a relatively small length in comparison to the first stopper tube body 135a but is not limited thereto and may include a hollow tube body formed by integrally forming the first and second stopper tube bodies through double injection molding.

The stopper 135 includes at least one rear moving tube 137 held and disposed due to a front end coming into contact with free ends of the pair of wing pieces 135c correspondingly inserted into an outer surface of the first stopper tube body 135a and spreading outward. The rear moving tube 137 may slide and move toward the front moving tube 128 coupled to the clip due to the insertion tube 110 entering along the linear guide path 141 of the cartridge.

The rear moving tube 137 may be formed as a hollow tube body inserted and disposed in an outer surface of a rear end of the first stopper tube body 135a to allow the wing piece 135c protruding from the outer surface of the first stopper tube body 135a to be contractibly deformed so as to coincide with the outer surface of the first stopper tube body and to be movable toward the clip 120.

The rear moving tube 137 corresponding to the wing piece 135c forms a tilted surface 137a on an inner circumferential surface of a front end so as to elastically deform the free end of the wing piece inward when moving toward the front moving tube 128 and be moved forward to the front moving tube 128 along the linear guide path.

It has been described that a contact end 137b such as an annular step corresponding to an outer diameter of the insertion tube 110 is formed on an inner circumferential surface of a rear end of the rear moving tube 137 corresponding to a front end of the insertion tube 110 so that the front end of the insertion tube 110 entering one open end of the linear guide path of the cartridge and a contact end of the rear moving tube come into contact with each other and an external force is applied. However, the present invention is not limited thereto and the front end of the insertion tube 110 entering the cartridge 140 may come into direct contact with the perpendicular rear end of the rear moving tube 137 and apply the external force.

A coupling step 131f protruding to be correspondingly coupled to a coupling groove 135f formed in a rear end of the stopper 135 is provided on the second outer diameter portion 131e corresponding to the second connector 131b.

Accordingly, through insertion-assembling caused by mutual coupling between the coupling groove 135f of the stopper 135 and the coupling step 131f of the clip connection body 131, the stopper 135 and the clip connection body 131 may be coupled to each other to make internal rotation of the clip connection body 131 disposed in the guide hole of the stopper difficult.

As shown in FIGS. 1, 2, and 3, the cartridge 140 is a structure in which the clip 120 including the pair of tong pieces 121a and 121b, the clip operating portion 130 including the clip connection body 131 having one end connected to the clip 120, and the moving tube separation bar 144 are internally arranged together and temporarily accommodated to allow the linker 113 and the clip connection body 131 to be elastically linked-connectable in the insertion tube 110 internally entering.

The cartridge 140 includes first and second divided covers 140a and 140b on inner surfaces facing each other which recessively form a linear guide groove having an approximately semicircular cross-sectional shape to form the linear guide path 141 of a circular hole having an approximately circular cross-sectional shape while being shape-matched.

A plurality of assembling pins 149 or assembling grooves which are correspondingly assembled while being shape-matched and generating a coupling force may be formed to protrude or to be recessed from an inner surface of any one of the first and second divided covers 140a and 140b and corresponding assembling pins or assembling grooves are formed on the other divided cover to be shape-matched through coupling therebetween, the present invention is not limited thereto and they may be shape-matched and assembled with each other using a variety of coupling methods.

On the linear guide path 141 formed inside the body of the cartridge 140, the clip 120 that is temporarily restricted by a retracted state of the pair of tong pieces 121a and 121b due to the front moving tube 128, the clip operating portion 130 including the rear moving tube 137 assembled with the outer surface of the rear end of the first stopper tube body 135a, and the moving tube separation bar 144 having an end facing the clip are arranged.

Accordingly, the clip 120 coupled to the front moving tube 128 is aligned and disposed while being retracted on the linear guide path 141 to be located between the clip operating portion 130 and the moving tube separation bar 144.

Here, respective outer diameters of the front and rear moving tubes 128 and 137 may be provided to be relatively smaller than or equal to an inner diameter of the linear guide path 141, and respective inner diameters of the front and rear moving tubes 128 and 137 may be provided to be approximately equal to a separation sensing portion 144a of the moving tube separation bar which has a maximum outer diameter.

The front moving tube 128 which restricts the pair of tong pieces 121a and 121b of the clip 120 in the retracted state and the rear moving tube 137 coupled to the first stopper tube body 135a of the stopper 135 have been described and shown as being provided as hollow tube bodies having inner and outer diameters and lengths approximately equal to each other but are not limited thereto and may be provided as hollow tube bodies having equal inner and outer diameters and different lengths.

A front end of the front moving tube 128 may be partially inserted into an end of the moving tube separation bar 144 and wait to be disposed on the linear guide path 141 to allow the front end of the clip 120 temporarily restricted in the retracted state to be in place at a center of the end of the moving tube separation bar 144 but is not limited thereto and the front moving tube 128 coupled to the clip may be spaced at a certain interval apart from the end of the moving tube separation bar 144 and wait to be disposed on the linear guide path 141.

A tapered inlet end 141a having a cross-sectional shape having an inner diameter gradually increasing toward the outside is formed on an opening end of the linear guide path 141 corresponding to the end of the insertion tube 110, and the moving tube separation bar 144 configured to collect the front and rear moving tubes 128 and 137 separated and moved from the clip and the clip operating portion due to the external force of the insertion tube entering is included on the other side of the inlet end 141a.

The moving tube separation bar 144 may be provided as a cylindrical body including the separation sensing portion 144a which has a certain length and the same inner diameter as the inner diameters of the front and rear moving tubes so as to generate a frictional force having a certain intensity when coming into contact with the inner surfaces of the front and rear moving tubes 128 and 137 and including a column portion 144b which has a certain length and an outer diameter relatively smaller than the inner diameters of the front and rear moving tubes and picks up the front and rear moving tubes 128 and 137 passing through the separation sensing portion due to an external force.

The moving tube separation bar 144 may include a fixed body 144d integrally connected to the other end of the column portion 144b to place the separation sensing portion 144a in position at the center of the linear guide path 141 and detachably position-fixed to any one side of the first and second divided covers.

The fixed body 144d has been shown and described as including a boss hole 144c press-fit assembled with a fixing boss 144e formed to protrude from a sealed end of the linear guide path 141 and detachably position-fixed but is not limited thereto and may be integrally provided with any one of the first and second divided covers of the cartridge.

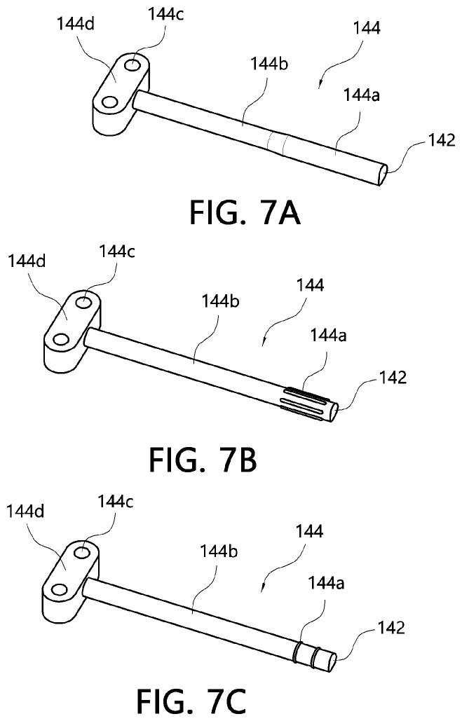

Meanwhile, as shown in FIGS. 7A, 7B, and 7C, the moving tube separation bar 144 includes a notch portion 142 forming an open angle spreading outward by a certain angle at an end of the separation sensing portion 144a corresponding to the front end of the clip 120 retracted by the front moving tube 128.

The notch portion 142 may be formed to be recessed with an inward concave arch shape or be incised with an approximately V-shape. The front ends of the pair of tong pieces restricted in the retracted state and collected by the front moving tube 128 come into precise contact with the notch portion 142 so as to restrict and limit forward movement of the clip when entering the insertion tube while allowing forward movement of the front and rear moving tubes toward the moving tube separation bar.

That is, while the serration portion that is the front end of the clip 120 and the notch portion 142 that is the end of the moving tube separation bar 144 come into contact with each other and restrict forward movement of the clip, the front moving tube 128 and the rear moving tube 137 are relatively moved by the external force of the insertion tube entering the linear guide path through the separation sensing bar 144a of the moving tube separation bar 144 to the column portion 144b and separately collected.

In the clip 120 from which the front moving tube 128 is separated and removed, the pair of tong pieces 121a and 121b enter and are disposed on the inner surface of the insertion tube 110 moved forward along the linear guide path 141 in order to move the rear moving tube 137 to a location of the separation sensing bar 144a and to separate the rear moving tube 137 therefrom while the front end of the clip comes into contact with the separation sensing bar.

Accordingly, the clip 120 is located in the insertion tube entering along the linear guide path 141 formed in the cartridge 140 to be restricted again and maintained equally to being restricted in the retracted state by the front moving tube 128.

Meanwhile, as shown in FIG. 7A, the separation sensing portion 144a may have an outer diameter that is relatively greater than an outer diameter of the column portion 144b to come into contact with the respective inner surfaces of the front and rear moving tubes 128 and 137 moved along the linear guide path 141 and generate a frictional force and may be provided as a cylindrical shape having an outer diameter that is relatively greater than or equal to the respective inner diameters of the front and rear moving tubes 128 and 137.

As shown in FIG. 7B, the separation sensing portion 144a may include at least one linear protruding rib extending with a certain length in a movement direction of the front moving tube 128 or the rear moving tube 137 to allow the respective inner surfaces and outer surfaces of the front and rear moving tubes 128 and 137 to come into partial contact with each other and generate a frictional force.

As shown in FIG. 7C, the separation sensing portion 144a may include at least one arc-shaped protruding rib intersecting with or perpendicular to the movement direction of the front moving tube 128 or the rear moving tube 137 to allow the respective inner surfaces of the front and rear moving tubes 128 and 137 to come into contact with each other and generate a frictional force.

The arc-shaped protruding rib has been illustrated and described as being continuously formed in a circumferential direction but is not limited thereto and may be discontinuously formed.

Accordingly, since the external force caused by the frictional force generated by contact between the separation sensing portion 144a and the front moving tube 128 or the rear moving tube 137 is transferred to a manipulator through the insertion tube and is indirectly recognizable by the manipulator during a process in which the front moving tube 128 and the rear moving tube 137 are moved and separately collected to the column portion 144b via the separation sensing portion 144a along the linear guide path, the manipulator moving the insertion tube forward may sensately check whether the front moving tube and the rear moving tube located on the column portion are separated and stop further excessive entering of the insertion tube.

In the endoscopic treatment device 100 including the above components, during a process in which a user inserts the insertion tube 110 along the linear guide path 141 of the cartridge, the linker 113 including the first and second linkers and the clip connection body 131 of the clip operating portion 130 are connected to each other in the insertion tube and then withdrawn outward from the cartridge 140.

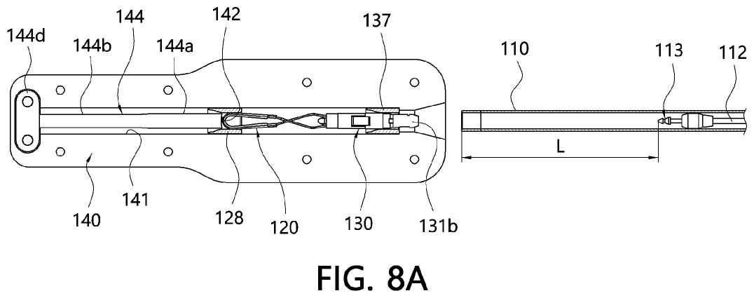

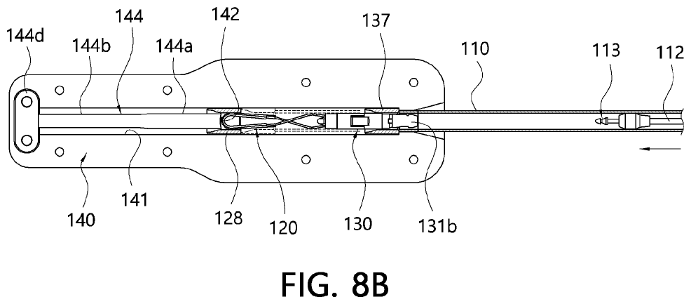

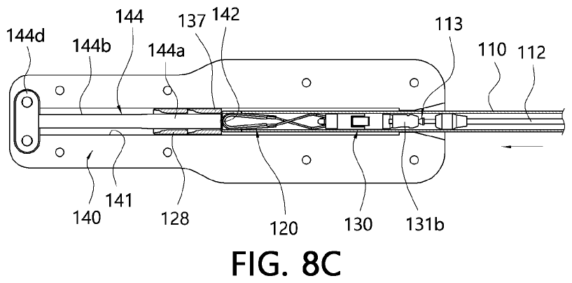

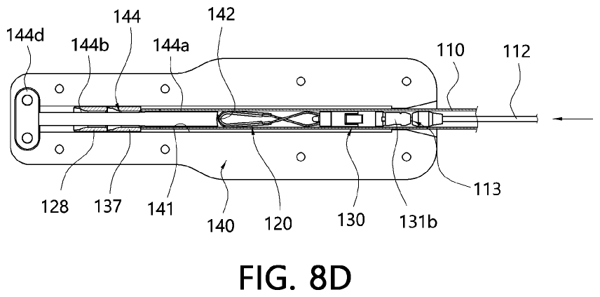

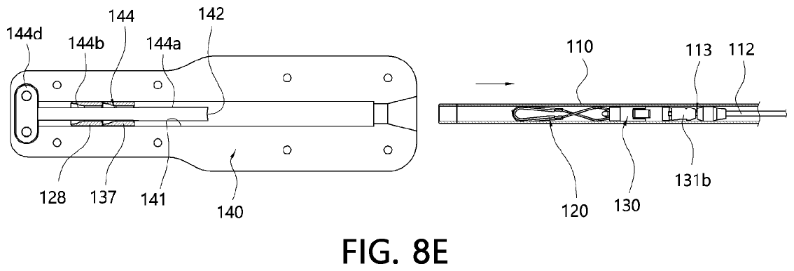

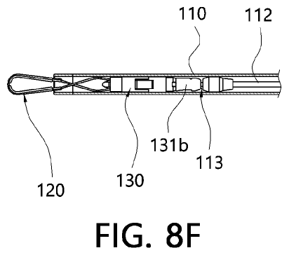

First, as shown in FIG. 8A, the clip 120 temporarily restricted in the retracted state by the front moving tube 128, the clip operating portion 130 including the clip connection body 131 having the front end connected to the clip, and the moving tube separation bar 144 including the front end corresponding to the clip 120 temporarily restricted in the retracted state are aligned and arranged on the linear guide path 141 linearly formed in the cartridge 140.

The front moving tube 128 which restricts and maintains the clip 120 in the retracted state is disposed in the linear guide path 141 so that a center of the front moving tube and a center of the linear guide path coincide with each other and the front end of the clip restricted in the retracted state by the front moving tube and the center of the moving tube separation bar correspond to each other.

The rear moving tube 137 assembled with the rear end of the stopper of the clip operating portion 130 is disposed in the linear guide path 141 and connected to the clip so that the clip connection body disposed in the stopper coincides with the center of the linear guide path.

The linker 113 enters to a certain length L inward from an open inlet end of the insertion tube 110 coming into contact with the rear end of the rear moving tube and stops and stands by.

Here, the certain length L setting a location of the linker 113 which stops and stands by inside the insertion tube 110 may be preset to be approximately equal to a distance between the end of the moving tube separation bar 144 with which the front end of the clip 120 comes into contact and the rear end of the clip connection body 131 with which the linker 113 comes into contact so that the front and rear moving tubes pass through the clip and are located in the moving tube separation bar 144 to allow the clip 120 to enter the insertion tube and be restricted again in the retracted state when the linker and the clip connection body are link-connected.

In this state, the open end of the insertion tube 110 is disposed to correspond to an open inlet end of the linear guide path 141.

As shown in FIGS. 8B and 8C, when the insertion tube 110 gripped by the user enters through an open inlet end 142a of the linear guide path 141, an end which is the front end of the insertion tube comes into contact with the rear end of the rear moving tube 137 and transfers an external force.

Due to the external force transferred through the insertion tube 110, the rear moving tube 137 is moved in one direction along the inside of the linear guide path 141 and passes through the stopper 135.

Here, the free end of the wing piece 135c protruding from the outer surface of the stopper 135 and spreading outward comes into contact with an inner surface of the rear moving tube 137, enters the first stopper tube body 135a, and is elastically deformed inward so as to be released from the rear moving tube.

The front end of the rear moving tube 137 passing through the stopper and forcibly moved to the clip due to the external force of the insertion tube 110 entering along the linear guide path 141 comes into contact with the rear end of the front moving tube 128 which restricts the clip in the retracted state to be moved with the front moving tube toward the moving tube separation bar.

As shown in FIG. 8C, when the front end of the insertion tube 110 entering in one direction along the inside of the linear guide path 141 passes by the clip 120 and is moved forward to the notch portion that is the end of the moving tube separation bar 144, the front moving tube 128 and the rear moving tube 137 including the front and rear ends coming into contact with each other pass the clip 120 and are located in the separation sensing portion 144a of the moving tube separation bar 144 due to the external force.

When the front and rear moving tubes 128 and 137 are forcibly moved toward the moving tube separation bar 144 and pass the clip, the clip 120 having the front end coming into contact with the notch portion 142 formed on the end of the moving tube separation bar 144 enters the insertion tube 110 and is restricted again in the retracted state.

A front end of the linker 113 standing by inside the insertion tube comes into contact with the second connector 131b that is the rear end of the clip connection body 131 and stands by for link-connection.

In this state, when forward movement of the insertion tube is continuously performed, as shown in FIG. 8D, an operation of connecting the first and second linkers of the linker 113 and the clip connection body of the clip operating portion 130 to each other in the insertion tube is performed.

When forward movement of the insertion tube is performed while the front end of the clip 120 comes into contact with the notch portion that is the end of the moving tube separation bar 144 and the front end of the linker 113 comes into contact with a rear end of the second connector 131b of the clip connection body 131, the linker 113 moved forward with the insertion tube is temporarily stopped in the insertion tube so that the linker enters while elastically spreading the pair of elastic holding pieces 133 provided on the second connector 131b both ways and is inserted and disposed in the hollow portion formed in the body of the second connector.

In this case, the first linker 113a is held by and connected to the internal step protruding from the inner surface of the hollow portion, and the second linker 113b is held by and connected to a protruding portion protruding from the inner ends of the pair of elastic holding pieces so that the clip connection body 131 and the linker 113 are link-connected to each other in the insertion tube entering along the linear guide path of the cartridge to forcibly move the front and rear moving tubes.

While the linker 113 and the clip connection body 131 are link-connected to each other, the front and rear moving tubes 128 and 137 are moved in position from the separation sensing portion 144a to the column portion 144b of the moving tube separation bar.

Meanwhile, whether the front and rear moving tubes 128 and 137 forcibly moved to the column portion of the moving tube separation bar 144 and separated are separated may be seen by the user sensately recognizing the external force caused by the frictional force generated between the outer surface of the separation sensing portion formed on the end of the moving tube separation bar and the inner surfaces of the front and rear moving tube which are targets to be separated using the cartridge or the insertion tube gripped by the user.

Also, when the front end of the insertion tube 110 is moved forward to the end of the separation sensing portion 144a while the front and rear moving tubes 128 and 137 are separately collected by the moving tube separation bar, the pair of tong pieces 121a and 121b of the clip 120 from which the front moving tube 128 is separated enter the insertion tube 110 so that the clip 120 is restricted and maintained by the insertion tube which has entered the cartridge as when restricted in the retracted state by the front moving tube 128 in the cartridge 140.

As shown in FIG. 8E, when the user withdraws the insertion tube 110 outward through the open inlet end of the linear guide path in the cartridge 140, the insertion tube 110 completely separated from the cartridge accommodates the clip operating portion 130 to which the linker 113 and the clip connection body are link-connected and the clip 120 connected thereto, and the front and rear moving tubes remain in the cartridge.

After the clip 120 separated from the cartridge and the clip operating portion 130 connected thereto are inserted with the insertion tube through the endoscopic channel, as shown in FIG. 8F, an endoscopic treatment operation of clamping and removing body tissue is performed using a clamping operation of the clip exposed from the open end of the insertion tube.

It will be apparent to those skilled in the art to which the present invention belongs that the present invention is not limited to the above-described embodiments and the attached drawings and various changes, substitutions, and alterations may be made herein without departing from the scope of the present invention.

1. A cartridge comprising a clip including a pair of tong pieces, a clip connection body having one end held by and connected to the clip, and a moving tube separation bar facing the clip, the cartridge comprising a linear guide path on which the clip connection body and the moving tube separation bar are linearly aligned and arranged,
wherein a front moving tube inserted and disposed in a front end of the clip facing the moving tube separation bar and configured to temporarily restrict the pair of tong pieces in a retracted state and a rear moving tube assembled with an outer surface of the clip connection body are arranged to be slidable and movable along the linear guide path toward the moving tube separation bar, and
wherein the clip is restricted again in the retracted state in the insertion tube entering along the linear guide path.

2. The cartridge of claim 1, wherein the front moving tube is disposed on the linear guide path while a front end thereof is partially inserted into an end of the moving tube separation bar or is disposed on the linear guide path while being spaced at a certain interval apart from the end of the moving tube separation bar.

3. The cartridge of claim 1, wherein the moving tube separation bar comprises a notch portion recessively formed in an end corresponding to the front end of the clip retracted by the front moving tube.

4. The cartridge of claim 1, wherein the moving tube separation bar comprises a separation sensing portion configured to generate a frictional force while outer surfaces and inner surfaces of the front moving tube and the rear moving tube passing therethrough, and a column portion in which the front and rear moving tubes passing through the separation sensing portion are disposed.

5. An endoscopic treatment device comprising:
an insertion tube including a wire including a linker on a front end;
a clip comprising a pair of tong pieces having front ends bent to face each other and intersecting with each other;
a clip operating portion comprising a clip connection body having one end held by and connected to the clip and a stopper with which the clip connection body is assembled to be reciprocable therein; and
a cartridge comprising a linear guide path, inside a body thereof, on which the clip connection body and a moving tube separation bar are linearly aligned and arranged,
wherein the endoscopic treatment device comprises a front moving tube inserted and disposed in a front end of the clip facing the moving tube separation bar and configured to temporarily restrict the pair of tong pieces in a retracted state and a rear moving tube assembled with an outer surface of the clip connection body,
wherein the front moving tube and the rear moving tube are allowed to slide and move by an external force of the insertion tube entering the cartridge along the linear guide path so as to be separately collected by the moving tube separation bar, and
wherein the linker and the clip connection body are link-connected to each other while the front end of the clip restricted again in the retracted state in the insertion tube comes into contact with an end of the moving tube separation bar.

6. The endoscopic treatment device of claim 5, wherein the linker comprises a first linker and a second linker adjacent thereto, and
wherein the clip connection body comprises a first connector held by and connected to the clip and a second connector link-connected to the linker.

7. The endoscopic treatment device of claim 6, wherein the second connector comprises a pair of elastic holding pieces divided by a slit line at an end corresponding to the first linker and a hollow portion in which the first and second linkers entering to elastically spread the pair of elastic holding pieces are inserted and disposed, and
wherein the first linker is held by and connected to an internal step protruding from an inner surface of the hollow portion and the second linker is held by and connected to a protruding portion protruding from inner ends of the pair of elastic holding pieces.

8. The endoscopic treatment device of claim 7, wherein the second linker comprises a concave groove forming the protruding portion and a tilted contact boundary surface in a rear surface corresponding to the protruding portion.

9. An endoscopic treatment device comprising:
an insertion tube including a wire including a linker on a front end;
a clip comprising a pair of tong pieces having front ends bent to face each other and intersecting with each other;
a clip operating portion equipped with a clip connection body comprising a connection bar configured to connect a first connector having one end held by and connected to the clip to a second connector having the other end link-connected to the linker so as to spread or retract the clip; and
a cartridge comprising a linear guide path, in a body thereof, on which the clip connection body and a moving tube separation bar are linearly aligned and arranged,
wherein the second connector comprises a pair of elastic holding pieces divided by a slit line at an end corresponding to the linker and a hollow portion in which the linker entering to elastically spread the pair of elastic holding pieces is inserted and disposed, and the linker is held by and connected to an internal step protruding from an inner surface of the hollow portion or is held by and connected to a protruding portion protruding from inner ends of the pair of elastic holding pieces.

10. The endoscopic treatment device of claim 9, wherein the linker comprises a first linker and a second linker adjacent thereto.

11. The endoscopic treatment device of claim 10, wherein the second connector comprises a pair of elastic holding pieces divided by a slit line at an end corresponding to the linker and a hollow portion in which the linker entering to elastically spread the pair of elastic holding pieces is inserted and disposed, and
wherein the first linker is held by and connected to an internal step protruding from an inner surface of the hollow portion or the second linker is held by and connected to a protruding portion protruding from inner ends of the pair of elastic holding pieces.

12. The endoscopic treatment device of claim 9, wherein the linker comprises a concave groove forming the protruding portion and a tilted contact boundary surface in a rear surface corresponding to the protruding portion.

13. The endoscopic treatment device of claim 9, wherein the linker enters to a certain length inward from an open inlet end of the insertion tube and stands by therein.

14. The endoscopic treatment device of claim 9, comprising:
a first incised portion incised and formed to externally expose the linker to one side in an outer surface of one side of the second connector; and
a second incised portion incised and formed to extend from the slit line and give an elastic force to the pair of elastic holding pieces in an outer surface of the other side of the second connector.

15. An endoscopic treatment device comprising:
an insertion tube including a wire including a linker on a front end;
a clip comprising a pair of tong pieces having front ends bent to face each other and intersecting with each other;
a clip operating portion equipped with a clip connection body comprising a connection bar configured to connect a first connector having one end held by and connected to the clip to a second connector having the other end link-connected to the linker so as to spread or retract the clip; and
a cartridge comprising a linear guide path, inside a body thereof, on which the clip connection body and a moving tube separation bar are linearly aligned and arranged,
wherein the endoscopic treatment device comprises a front moving tube inserted and disposed in a front end of the clip facing the moving tube separation bar and configured to temporarily restrict the pair of tong pieces in a retracted state and a rear moving tube assembled with an outer surface of the clip connection body,
wherein the second connector comprises a pair of elastic holding pieces divided by a slit line on an end corresponding to the linker and a hollow portion in which the linker entering to elastically spread the pair of elastic holding pieces is inserted and disposed,
wherein the front moving tube and the rear moving tube are allowed to slide and move by an external force of the insertion tube entering the cartridge along the linear guide path so as to be separately collected by the moving tube separation bar, and
wherein while the front end of the clip restricted again in a retracted state in the insertion tube comes into contact with an end of the moving tube separation bar, the linker is held by and connected to an internal step protruding from an inner surface of the hollow portion or is held by and connected to a protruding portion protruding from inner ends of the pair of elastic holding pieces so as to be link-connected to the clip connection body.
